(19) United States
(12) Patent Application Publication
(10) Pub. No.: US 2022/0256798 A1
(43) Pub. Date: Aug. 18, 2022

(54) VALUE-PHENOTYPED AUTOFLOWER CANNABIS PLANTS

(71) Applicant: Central Coast Agriculture, Inc., Buellton, CA (US)

(72) Inventors: Adam Criswell, Buellton, CA (US); Daniel Barrera, Buellton, CA (US); Steve Bobzin, Buellton, CA (US); John De Friel, Buellton, CA (US)

(21) Appl. No.: 17/651,310

(22) Filed: Feb. 16, 2022

Related U.S. Application Data
(60) Provisional application No. 63/150,381, filed on Feb. 17, 2021.

(51) Int. Cl. A01H 6/28; A01H 1/04; A01H 5/02; A01H 1/02

(52) U.S. Cl. CPC A01H 6/28; A01H 1/021; A01H 5/02; A01H 1/045

(57) ABSTRACT

The present invention relates to day-length neutral Cannabis plants with one or more value phenotypes.

### CLAIM OF PRIORITY UNDER 35 U.S.C. § 119

The present application for patent claims priority to Provisional Application No. 63/150,381 entitled “VALUE-PHENOTYPED AUTOFLOWER CANNABIS PLANTS” filed Feb. 17, 2021, which is hereby expressly incorporated by reference herein.

### BACKGROUND

### Field

The present invention relates to day-length neutral Cannabis plants with one or more value phenotypes.

### Background

“Autoflower” or “day-length neutral” Cannabis varieties are those that transition from a vegetative growth stage to a flowering stage based upon age, rather than day length. In contrast, most varieties of Cannabis in commercial use transition to the flowering stage based upon the plant's perception of day length, such that the plants flower according to the seasonal variation in day length rather than the developmental stage of the plant that coincides with age or more specifically degree days.

The autoflower trait in Cannabis plants can allow for a more consistent crop in terms of growth, yield, and harvest times as compared with day-length sensitive Cannabis varieties. In outdoor Cannabis cultivation, the availability of elite autoflower Cannabis varieties would expand the latitude and planting dates for productive Cannabis cultivation.

### SUMMARY

Some embodiments of the invention relate to a Cannabis plant or plant part with an Autoflower Value Phenotype, wherein the Autoflower Value Phenotype is seed-propagated, stable and uniformly expressed, wherein the Autoflower Value Phenotype comprises at least one value trait selected from: high THCA accumulation; specific cannabinoid ratio(s); a desirable composition of terpenes and/or other aromatic molecules; biomass yield; biomass composition; crude flower oil yield; crude flower oil composition; specific variants affecting cannabinoid or aromatic molecule biosynthetic pathways; a finished plant height that enables tractor farming inside high tunnels; modulators of the flowering time phenotype that increase or decrease maturation time; high flower to leaf ratios that enable pathogen resilience through improved airflow; high flower to leaf ratios that maximize light penetration and flower development in the vertical canopy space; a finished plant height and flower to leaf ratio that maximizes light penetration all the way to the ground but minimizes total plant height; and/or advantageous flower structures for oil or flower production (flower diameter, flower length, flower density, long or short internodal spacing distance, flower-to-leaf determination ratio of flower tissue (leafiness of flower), and/or uniform size, shape, and density of flowers throughout a plant.

In some embodiments, the plant has at least two value traits listed in paragraph [0005]. In some embodiments, the plant has at least 2, 3, 4, 5, or more value trails listed in paragraph [0005].

In some embodiments, a quantitative level of the value trait can be at least 70% of the value trait present in a photoperiod parent.

In some embodiments, a quantitative level of the value trait has not previously been available in seed-propagated autoflower Cannabis displaying substantial uniformity and stability as a seed line.

In some embodiments, the value trait can be high THCA accumulation, where the plant has THCA levels greater than 20%.

In some embodiments, the plant can have improved crude oil yield, where the crude oil yield can be at least 4.5%.

In some embodiments, the plant can have an advantageous plant structure, wherein the advantageous plant structure can permit a light measurement at a given position, and/or a total light measurement of all positions, at least 10% greater than a reference autoflower parent plant.

In some embodiments, the plant can have high THCA accumulation and advantageous flower structure.

In some embodiments, the plant can have high THCA accumulation and desirable terpene composition.

In some embodiments, the plant can have high crude oil yield and high biomass yield.

Some embodiments of the invention relate to a method of plant breeding to develop the plant disclosed herein. The method can include one or more of: (a) providing a first parent plant having a phenotype defined as a Value Phenotype, wherein the Value Phenotype comprises at least one trait of interest; providing a second parent plant, having an autoflower phenotype; (b) crossing the first and second parent plants; (c) recovering progeny from the crossing step; (d) screening the progeny phenotypically for presence of at least one autoflower allele and the Value Phenotype; (e) selecting autoflower carrier progeny with the Value Phenotype, wherein cells of said autoflower carrier progeny comprise at least one autoflower allele; (f) conducting further breeding steps using autoflower carrier progeny selfed, sib-mated, or crossed with plants having the Value Phenotype; and/or (g) repeating steps e, f, and g until at least one plant having an Autoflower Value Phenotype is obtained.

Some embodiments of the invention relate to a method of plant breeding to develop the plant disclosed herein, where the method can include one or more of: (a) providing a first parent plant, having a phenotype defined as a Value Phenotype, wherein the Value Phenotype comprises at least one trait of interest; (b) providing a second parent plant, having an autoflower phenotype; (c) crossing the first and second parent plants; (d) recovering progeny from the crossing step; (e) screening the progeny for presence of at least one autoflower allele using a marker having at least 51% correlation with presence of the autoflower allele or without a marker by screening the progeny's selfed offspring for the ability to produce the homozygous autoflowering phenotypes; (f) selecting autoflower carrier progeny, wherein cells of said autoflower carrier progeny comprise at least one autoflower allele; (g) conducting further breeding steps using autoflower carrier progeny crossed with plants having the Value Phenotype; and/or (h) repeating steps e, f, and g until at least one plant having an Autoflower Value Phenotype is obtained.

### BRIEF DESCRIPTION OF THE DRAWINGS

FIG. 1 shows results from QTL mapping

FIG. 2 is a box plot comparing oil yield in autoflowers (AF) vs. photoperiods (PP).

FIG. 3 is a box plot comparing cannabinoid concentrations in Autoflowers (AF) vs. Photoperiods (PP).

FIG. 4 is a box plot comparing terpene concentrations in Autoflowers (AF) vs. Photoperiods (PP).

FIG. 5 is a box plot comparing THC levels in Autoflowers (AF) vs. Photoperiods (PP).

### DETAILED DESCRIPTION

Day-length neutral (also referred to as autoflower or AF) Cannabis plants typically express less desirable phenotypic characteristics than day-length sensitive Cannabis. For example, lower cannabinoid content, leafy inflorescences and a limited aroma profile are commonly associated with day-length neutral (also referred to as photoperiod or PP) varieties, which tend to produce an inferior finished product. There is significant interest in breeding Cannabis to develop autoflower varieties that otherwise have desirable genotypes or phenotypes. Such breeding typically involves a cross of a first, day-length sensitive parent plant having a desired phenotype (referred to herein as a “Value Phenotype”) with a second parent plant having an autoflower phenotype, whatever other traits it may have. For purposes of this disclosure, a plant expressing all of the desirable features of a given first parent, the Value Phenotype, but in an autoflower form, can be referred to as an “Autoflower Value Phenotype” plant.

The present invention relates to Cannabis plants with a Value Phenotype and methods of producing the same. The plant can be stably seed-propagated.

The Value Phenotype can include at least one trait selected from one or more of: high THCA accumulation; specific cannabinoid ratio(s); a composition of terpenes and/or other aroma-active and aromatic molecules; monoecy or dioecy (enable or prevent hermaphroditism); branchless or branched architectures with specific height to branch length ratios or total branch length; determinant growth; time to maturity; high flower to leaf ratios that enable pathogen resistance through improved airflow; high flower to leaf ratios that maximize light penetration and flower development in the vertical canopy space; a finished plant height that enables tractor farming inside high tunnels; a finished plant height and flower to leaf ratio that maximizes light penetration all the way to the ground but minimizes total plant height; trichome size; trichome density; advantageous flower structures for oil or flower production (flower diameter length, long or short internodal spacing distance, flower-to-leaf determination ratio (leafiness of flower); metabolites that provide enhanced properties to finished oil products (oxidation resistance, color stability, cannabinoid and terpene stability); specific variants affecting cannabinoid or aromatic molecule biosynthetic pathways; modulators of the flowering time phenotype that increase or decrease maturation time; biomass yield and composition; crude oil yield and composition; resistance to botrytis, powdery mildew, fusarium, pythium, cladosporium, alternaria, spider mites, broad mites, russet mites, aphids, nematodes, caterpillars, HLVd or any other Cannabis pathogen or pest of viral, bacterial, fungal, insect, or animal origin; propensity to host specific beneficial and/or endophytic microflora; heavy metal composition in tissues; specific petiole and leaf angles and lengths; and/or the like.

For purposes of this disclosure, “stable” means that a given trait or property appears in repeated generations of the same cultivar, in a consistent manner. In some embodiments, where a given quantifiable trait or property appears at a given value in individuals or an aggregate of a given generation of a cultivar, the same quantifiable trait or property appears in another generation of the same cultivar within 85% 90%, 92%, 95%, 98%, 99%, 100%, 101%, 102%, 105%, 108%, 110%, or 115% of the value manifest in the previous generation.

For purposes of this disclosure, “uniform” means that a given trait or property appears consistently among individuals within a given population of a cultivar. In some embodiments, where a given quantifiable trait or property appears at a given average value in a given population of a cultivar, 85% 90%, 92%, 95%, 98%, 99%, or more, of the individuals in the population manifest such trait or property within 85% 90%, 92%, 95%, 98%, 99%, 100%, 101%, 102%, 105%, 108%, 110%, or 115% of the average value of the population.

### Value Phenotype and Autoflower Value Phenotype

The Value Phenotype can include but is not limited to, at least one of the traits listed in paragraph [0005] in a desirable form. In some embodiments, an Autoflower Value Phenotype can be defined as an Autoflower plant displaying selected Value Phenotype traits determined to be desirable (referred to as “Target Value Traits”), within 70%, 75%, 80%, 85%, 90%, 91%, 92%, 93%, 94%, 95%, 96%, 97%, 98%, 99%, 99.5%, or more, of each of the Target Value Traits present in the photoperiod parent in the original cross. Accordingly, in these embodiments, definition of an Autoflower Value Phenotype is in quantitative reference to the day-length-sensitive (photoperiod) parent, rather than being in reference to a more general set of values for one or more Target Value Traits that are not, themselves, based upon or defined by the genetic background of the Value Phenotype photoperiod parent. In other embodiments, the quantitative levels of all Target Value Traits in the Autoflower Value Phenotype plant have not previously been available in seed-propagated autoflower Cannabis displaying substantial uniformity and stability as a seed line.

### High THCA Accumulation

In some embodiments, the plant can have THCA levels of greater than 20%, 22%, 24%, 26%, 28%, 30%, 32%, 34%, 36%, 38%, 40% or more weight percent dry flower. THCa is the primary psychoactive component in Cannabis. The potency of THCa in Cannabis flower is one of the primary metrics of quality for the consumer of recreational Cannabis flower. Higher THCa potency is also valuable for processing Cannabis flower to oil, concentrates, and other products by reducing the amount, time, and effort of processing flower to concentrates or isolate of THCa.

### High Amounts of Other Cannabinoids

In some embodiments, the plant can express levels of one or more other cannabinoids, with or without THC, such that the amount of a given single cannabinoid or the combined amount of two or more cannabinoids (the “Relevant Cannabinoid(s)”), exceeds that which previously has been observed in plants having an autoflower phenotype derived from a cross with a given photoperiod Value Phenotype parent. Accordingly, such Autoflower Value Phenotype plant can express a level of the Relevant Cannabinoid(s) within 70%, 75%, 80%, 85%, 90%, 91%, 92%, 93%, 94%, 95%, 96%, 97%, 98%, 99%, 99.5%, or more, the same Relevant Cannabinoid(s) found in the original photoperiod Value Phenotype parent. In other embodiments, the total amount of THCA and/or any other Relevant Cannabinoid(s) have not previously been available in seed-propagated autoflower Cannabis displaying substantial uniformity and stability as a seed line. The cannabinoids can be, but are not necessarily limited to: tetrahydrocannabinolic acid A (THCA-A), tetrahydrocannabinolic acid B (THCAB), tetrahydrocannabinol (THC), tetrahydrocannabinolic acid C (THCA-C), tetrahydrocannabinol C (THC-C), tetrahydrocannabivarinic acid (THCVA), tetrahydrocannabivarin (THCV), tetrahydrocannabiorcolic acid (THCA-C), tetrahydrocannabiorcol (THC-C), delta-7-cis-iso-tetrahydrocannabivarin, delta-8-tetrahydrocannabinolic acid (Δ8-THCA), delta-9-tetrahydrocannabinol (Δ9-THC), cannabidiolic Acid (CBDA), cannabidiol (CBD), cannabidiol monomethylether (CBDM), cannabidiol-C(CBD-C), cannabidivarinic acid (CBDVA), cannabidivarin (CBDV), cannabidiorcol (CBD-C), cannabigerolic acid (CBGA), cannabigerolic acid monomethylether (CBGAM), cannabigerol (CBG), cannabigerol monomethylether (CBGM), cannabigerovarinic Acid (CBGVA), cannabigerovarin (CBGV), cannabichromenic Acid (CBCA), cannabichromene (CBC), cannabichromevarinic Acid (CBCVA), cannabichromevarin (CBCV), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabivarin (CB V), cannabinol-C(CBN-C), cannabiorcol (CBN-C1), cannabinodiol (CBND), cannabinodivarin (CB VD), cannabitriol (CBT), 10-Ethoxy-9-hydroxy-delta-6a-tetrahydrocannabinol, 8,9-dihydroxy-delta-6a-tetrahydrocannabinol (8,9-Di-OH-CBT-C5), cannabitriolvarin (CBTV), ethoxy-cannabitriolvarin (CBTVE), dehydrocannabifuran (DCBF), cannabifuran (CBF), cannabichromanon (CBCN), cannabicitran (CBT), l0-oxo-delta-6a-tetrahydrocannabinol (OTHC), delta-9-cis-tetrahydrocannabinol (Δ9-cis-THC), cannabiripsol (CBR), -3,4,5,6-tetrahydro-7-hydroxy-alpha-alpha-2-trimethyl-9-n-propyl-2,6-methano-2H-1-benzoxocin-5-methanol (OH-iso-HHCV), trihydroxy-delta-9-tetrahydrocannabinol (triOH-THC), an isocanabinoid, any other cannabinoid, and any combination thereof.

### Specific Cannabinoid Ratio(s)

In some embodiments, the Autoflower Value Phenotype plant can have cannabinoid ratio(s) of advantageously selected for specific uses and/or products, wherein such cannabinoid ratios have not previously been available in seed-propagated autoflower Cannabis displaying substantial uniformity and stability as a seed line. In other embodiments, the cannabinoid ratios are within 70%, 75%, 80%, 85%, 90%, 91%, 92%, 93%, 94%, 95%, 96%, 97%, 98%, 99%, 99.5%, or more, the corresponding cannabinoid ratios found in the original photoperiod Value Phenotype parent.

A Composition of Terpenes and/or Other Aromatic Molecules

In some embodiments, the plant can have one or more terpenes and/or other aromatic molecules including, for example, a-bisabolol, borneol, camphene, camphor, 3-carene, caryophyllene oxide, b-caryophyllene, a-cedrene, citronellol, p-cymene, eucalyptol, fenchol, geraniol, geranyl acetate, guaiol, a-humulene, isobomeol, (−)-isopulegol, limonene, linalool, menthol, myrcene, nerolidol, ocimene, phellandrene, phytol, a-pinene, b-pinene, R-(+)-pulegone, sabinene, a-terpinene, terpinen-4-ol, a-terpineol, 4-terlineol, terpinolene, valencene, or the like; or apigenin, cannflavin A, cannflavin B, cannflavin C, chrysoeriol, cosmosiin, flavocannabiside, kaempferol, luteolin, myricetin, orientin, isoorientin (homoorientin), quercetin, (+)-taxifolin, vitexin, and isovitexin, and/or the like. Other molecules can include, but are not limited to, compounds from the following table.

TABLE 1

| | CAS | |
|---|---|---|
| Compound Name | Number | Descriptor |
| 2,3,5-trimethylpyrazine | 14667-55-1 | Roasted |
| 2-Ethyl-3-Methylpyrazine | 15707-23-0 | Nutty |
| 2-Ethyl-3(5 or 6)-dimethylpyrazine | 27043-05-06 | Nutty |
| Butyric Acid | 107-92-6 | Cheese |
| Furfuryl Mercaptan (2- | 98-02-2 | Sulphur, coffee |
| furanmethanethiol) | | |
| 4-Methylthio-4-methyl-2-pentanone | 23550-40-5 | Sulphur |
| cis-3-Hexen-1-ol | 928-96-1 | Green, grass |
| 2-Isobutyl-3-methoxypyrazine | 24683-00-9 | Green, bell pepper |
| 2-Isopropyl-3-methoxypyrazine | 25773-40-4 | Green, bell pepper |
| Methyl butyrate | 623-42-7 | Fruity |
| Ethyl butyrate | 105-54-4 | Fruity |
| Ethyl 2-methylbutyrate | 7452-79-1 | Fruity |
| Ethyl isovalerate | 108-64-5 | Fruity |
| Hexanal | 66-25-1 | Green, Grassy |
| Phenethyl acetate | 103-45-7 | Rose, Floral, |
| | | Honey, Sweet |
| Ethyl phenylacetate | 101-97-3 | Floral, Honey, |
| | | Balsamic |
| 2-sec-Butyl-3-methoxypyrazine (2- | 24168-70-5 | Musty, Green, |
| Methoxy-3-(1- | | Bell pepper |
| methylpropyl)pyrazine) | | |
| Methyl Isovalerate | 556-24-1 | Fruity |
| 1-Octen-3-one | 4312-99-6 | Mushroom |
| p-Anisaldehyde | 0123-11-5 | Anise |
| Phenethyl alcohol | 60-12-8 | Floral |
| 2′-Aminoacetophenone | 551-93-9 | Grape, floral |
| trans-Anethole | 4180-23-8 | anise, licorice |
| 3-Methylbut-2-ene-1-thiol | 5287-45-6 | Skunk |
| Hexyl acetate | 142-92-7 | Banana |

The composition of terpenes and/or other aromatic molecules can contribute to desirable characteristic such as a desirable taste or smell. For example, the plant can have one or more of the following aroma descriptors: Ammonia, Apple, Apricot, Armpit, Banana, Berry, Blueberry, Bright, Bubblegum, Butter, Candy, Cheese, Chemical, Chlorine, Citrus, Coffee, Cough Syrup, Creamy, Deep, Dough, Earthy, Floral, Fresh, Fruit, Funk, Gas, Gelato, Gnarly, Grape, Grapefruit, Gross, Heavy, Herbal, Kush, Lavender, Lemon, Lemonade, Light, Lime, Low, Mango, Melon, Menthol, Mint, Mothball (Camphor), Neutral, Oily, Orange, Peach, Pear, Pepper, Pine, Pineapple, Punch, Rose, Rotten, Sage, Sharp, Skunk, Soapy, Sour, Spice, Spicy, Strawberry, Strong, Sweet, Tar, Terpinolene, Tobacco, Vanilla, Weird, Woody, Zest, and/or the like, and/or any combination thereof.

In some embodiments, the Autoflower Value Phenotype plant can have a profile and/or abundance of one or more terpenes and/or other aromatic molecules that have not previously been available in seed-propagated autoflower Cannabis displaying substantial uniformity and stability as a seed line. In other embodiments, the profile and/or abundance of one or more terpenes and/or other aromatic molecules are within 70%, 75%, 80%, 85%, 90%, 91%, 92%, 93%, 94%, 95%, 96%, 97%, 98%, 99%, 99.5%, or more, the corresponding profile and/or abundance of one or more terpenes and/or other aromatic molecules found in the original photoperiod Value Phenotype parent.

### Biomass Yield

In some embodiments, the plant can have an improved biomass yield as compared with biomass yields previously available in an autoflower seed-propagated Cannabis variety displaying substantial uniformity and stability as a seed line. In some embodiments, biomass yield can be defined as flower biomass only (i.e., excluding stalks and most leaves). In other embodiments, biomass can be defined as whole plant biomass.

In some embodiments, the biomass is within 70%, 75%, 80%, 85%, 90%, 91%, 92%, 93%, 94%, 95%, 96%, 97%, 98%, 99%, 99.5%, or more found in the original photoperiod Value Phenotype parent. Biomass can be quantified on a per plant or per acre basis. For example, the biomass can be 750, 800, 850, 900, 950, 1000, 1250, 1500 or more grams per plant. For example, the biomass can be 10,000; 15,000; 20,000; 25,000; 30,000; 35,000; or more pounds per acre.

### Biomass Composition

In some embodiments, the plant can have a biomass composition advantageously selected for specific uses and/or products, wherein such composition has not previously been available in seed-propagated autoflower Cannabis displaying substantial uniformity and stability as a seed line. Biomass composition in this context can be any composition profile is substantially suited for a particular desired use of the Cannabis plant. Where biomass is defined as the total biological tissue above the level of soil or other substrate in which a plant is grown, such biological tissue can be subdivided into inflorescences (which can be subdivided into floral tissue and leafy tissue); non-inflorescence leaves; and stem and branch tissue to which leaves are attached. In embodiments that divide biomass composition into these morphological groups, a desirable composition for production of extracts is one in which floral tissue is at least one standard deviation above the average floral tissue of the AF parent. Likewise, a desirable composition for fiber production is one in which stem and branch tissue is at least one standard deviation above the average stem and branch tissue of the AF parent. Further, a desirable composition for extraction of culinary products including seeds, seed oils, and/or seed proteins is one in which seed set is at least one standard deviation above the average seed set of the AF parent. In some embodiments, the floral tissue, stem and branch tissue, and/or seed set is at least 1, 2, 3, 4, 5, 10, 15, 20, 25% or more above the average of the AF parent.

### Crude Oil Yield

In some embodiments, the plant can have an improved crude oil yield as compared with crude oil yields previously available in an autoflower seed-propagated Cannabis variety displaying substantial uniformity and stability as a seed line. For purposes of this disclosure, “crude oil” refers to oil extracted from flower tissue and/or from trichomes, and is not intended to be inclusive of seed oils. In some embodiments, crude oil yield can be at least 20, 25, 30, 35, 40, 45, 50% or more compared to the AF parent.

Crude oil yield average for AF is 3.15% and for PP is 5.9%. Thus, any value above 3.15% can be considered improved yield for an autoflower. For example, improved yield could be 4.1, 4.2, 4.3, 4.4., 4.5., 4.6, 4.7, 4.8, 4.9, 5.1, 5.2, 5.3, 5.4, 5.5, or more %.

### Crude Oil Composition

In some embodiments, the plant can have a crude oil composition advantageously selected for specific uses and/or products, wherein such composition has not previously been available in seed-propagated autoflower Cannabis displaying substantial uniformity and stability as a seed line.

### Specific Variants Affecting Cannabinoid or Aromatic Molecule Biosynthetic Pathways

In some embodiments, the plant can include specific variants that can affect biosynthetic pathways of cannabinoids and/or other aromatic molecules.

### A Desirable Finished Plant Height

In some embodiments, the height can enable tractor farming inside high tunnels. In some embodiments, the height can maximize light penetration all the way to the ground but minimizes total plant height. The height can be about 1, 2, 3, 6, 12, 24, 36, 48, 60, inches of more.

In some embodiments, the height is within 70%, 75%, 80%, 85%, 90%, 91%, 92%, 93%, 94%, 95%, 96%, 97%, 98%, 99%, 99.5%, or more, the corresponding height found in the original photoperiod Value Phenotype parent or the AF parent.

### Modulators of the Flowering Time Phenotype

In some embodiments, the plant can include modulators of the flowering time, or days to maturity, phenotype. A modulator can be a gene that affects the timing of maturation. The modulators can increase or decrease maturation time, typically resulting in maturation at any of 50 to 120 days after sowing. In some embodiments, modulators are selected which permit the shortest time to reach maturity with sufficient biomass accumulation. In some embodiments, a longer flowering time can be useful to permit further vegetative growth while still having substantially synchronous flowering. In various embodiments, a variety of maturation times can be selected by selecting for the appropriate modulators thereof and/or by selecting for progeny of a cross that mature at the desired number of days after sowing. In some embodiments, maturation time can be about 60-110 days after sowing. For example, maturation time can be about 60, 65, 70, 75, 80, 85, 90, 95, 100, or 110 days.

### High Flower to Leaf Ratio

In some embodiments, the plant can include a flower to leaf ratio not previously available in an autoflower seed-propagated Cannabis variety displaying substantial uniformity and stability as a seed line. In some embodiments, the flower to leaf ratio can enable pathogen resilience through improved airflow. In some embodiments, the flower to leaf ratio can maximize light penetration and flower development in the vertical canopy space. In various embodiments these properties can be measured by weighing total leaf tissue versus total flower tissue upon harvest of randomly selected members of a population and establishing a baseline ratio, and then comparing such ratio to members of other populations that have been selected for a different ratio. In some embodiments, the invention provides a shift in the ratios of at least 10%. For example, the invention can provide a shift in the ratio of about 10%, 15%, 20%, 25%, 30%, 35%, 40%, 45%, 50%, or more.

In other embodiments, airflow improvements can be measured by selecting plants randomly and blowing a gas stream of specified dimensions and at specified positions on the plant, and detecting velocity, turbulence, and/or other properties of the gas stream on the opposite side of the plant, to quantify the plant's resistance to airflow. Such an assay can be directed horizontally, vertically, or at any angle as desired. Having quantified a baseline for a given plant, other plants can be tested using the same parameters and then quantitatively compared. In some embodiments thus compared, the invention provides a difference in terms of resistance to airflow or other properties of airflow, of at least 10%. For example, the invention can provide a difference of about 10%, 15%, 20%, 25%, 30%, 35%, 40%, 45%, 50%, or more.

In still other embodiments, where improved plant structure is a function of maximizing light penetration, certain positions of a plant are defined in refence to a given internode, height above the substrate surface, distance from the apex, and the like, and light reaching such positions is quantified and compared with light reaching a meter just above the top of the plant. The standard light penetration at such reference points is then scored and compared with other plants having been selected for improved light-penetration structure. In some embodiments, the invention provides an AF plant whose structure permits a light measurement at a given position, and/or a total light measurement of all positions, at least 10% greater than the reference AF plant. For example, the invention can provide an AF plant whose structure permits a greater light measurement at a given position, and/or a greater total light measurement of about 10%, 15%, 20%, 25%, 30%, 35%, 40%, 45%, 50%, or more, in comparison with the reference AF plant or plants.

### Advantageous Flower Structures for Oil or Flower Production

In some embodiments, the plant can include advantageous flower structures for oil or flower production including, but not limited to, specifically targeted flower diameters, internodal spacing distances, and/or flower-to-leaf determination ratios (leafiness of flower) not previously available in an autoflower seed-propagated Cannabis variety displaying substantial uniformity and stability as a seed line. For example, variations in floral structure, depending upon the desired use of the flower, can include a large number of small inflorescences or, in contrast, a smaller number of large inflorescences. Likewise, variations in floral structure can include highly compact, dense flower arrangement or, in contrast, open flower arrangement. In addition, floral structures can be inflorescences with numerous inflorescence leaves (sugar leaves) or, in contrast, inflorescences can be nearly leafless.

In still other embodiments the relevant aspect of flower structure can be the number, density, and/or morphology of the trichomes found on floral tissue. Trichomes can range from sparse to dense, with dense typically being favored in order to obtain greater yields of crude oil. Trichomes can also vary from large to small, again with large generally being favored. An aspect of trichome morphology for which variations can be desirable is the fragility of the stalk structure connecting the glandular head of the trichome to the basal cells or leaf issue. In some cases, a fragile stalk can be desirable for ease of direct trichome harvest while, in contrast, it can be desirable for the stalk to resist breakage to avoid trichome loss during flower handling.

Accordingly, any or all of these variations can be desirable for certain uses and not for others, and some compatible combinations of these variations can also be desirable such as, for example, large inflorescences with loose flowers and numerous trichomes having fragile stalks. Such a morphology can enhance the ease and efficiency of direct harvest of trichomes, while other morphological variations can be more advantageous for other uses. Quantification of flower morphology can take many forms depending upon the most desired factors and is within the level of skill in the art. Embodiments of the invention provide autoflower progeny-line plants selected for advantageous flower structures, however quantified, being at least 10% greater than the original AF parent of the progeny line. For example, the advantageous flower structures, however quantified, can be about 10%, 15%, 20%, 25%, 30%, 35%, 40%, 45%, 50%, or more greater than the original AF parent of the progeny line.

### Breeding Methods

Some embodiments of the invention relate to breeding methods used to make the plant or plant part. Breeding methods that can be used in certain embodiments of the invention, can be found, for example in, U.S. patent Ser. No. 10/441,617B2, which is incorporated herein by reference.

An Autoflower Value Phenotype inbred line (or hybrid or population) can be developed using the techniques of backcrossing, selfing, sib-mating, and/or dihaploids, or any other technique used to develop parental lines in plant breeding.

In a method of backcrossing, the autoflower trait can be introgressed into a parent having the Value Phenotype (the recurrent parent) by crossing a first plant of the recurrent parent with a second plant having the autoflower trait, but differing from the recurrent parent and being referred to herein as the “donor parent’. The recurrent parent is a plant that does not have the autoflower trait but possesses a Value Phenotype. The progeny resulting from a cross between the recurrent parent and donor parent is referred to as the F1 progeny. One or several plants from the F1 progeny can be backcrossed to the recurrent parent to produce a first-generation backcross progeny. One or several plants from the first-generation backcross progeny can be backcrossed to the recurrent parent to produce a second-generation backcross progeny. This process can be performed for one, two, three, four, five, or more generations. At each generation including the F1 progeny, the first-generation backcross progeny, and all subsequent generations, the population can be screened for the desired characteristics, which screening can occur in a number of different ways. For instance, the population can be screened using phenotypic screens or quantitative bioassays as known in the art, which phenotypic screens or bioassays may be performed on individual plants of the population or on selfed or crossed progeny from the same individual plants. Alternatively, instead of using bioassays, marker-assisted selection can be performed to identify those plants that contain the autoflower trait. Following screening, at each generation, one or several plants can be selected that contain the autoflower trait as determined through either phenotypic or, in some embodiments, genotypic screening, and backcrossed to the recurrent parent for a number of generations in order to allow for the cannabis plant to become increasingly similar to the recurrent parent. This process can be performed for one, two, three, four, five, or more generations. In principle, the progeny resulting from the process of crossing the recurrent parent with the autoflower donor parent are heterozygous for one or more genes responsible for autoflowering. When appropriate the last backcross generation can be selfed in order to provide for homozygous pure breeding (inbred) progeny with Autoflower Value Phenotype.

In a method of selfing, the autoflower trait can be introgressed into a parent having the Value Phenotype (the Value Phenotype parent) by crossing a first plant of the Value Phenotype parent with a second plant having the autoflower trait, but differing from the Value Phenotype parent and being referred to herein as the “autoflower parent’. The Value Phenotype parent is a plant that does not have the autoflower trait but possesses a Value Phenotype. The progeny resulting from a cross between the Value Phenotype parent and autoflower parent is referred to as the F1 progeny. One or several plants from the F1 progeny can be self-fertilized or cross-fertilized (sib-mated) to produce a first-generation selfed or sib-mated progeny. One or several plants from the first-generation selfed or sib-mated progeny can be selfed or sib-mated again to produce a second-generation selfed or sib-mated progeny. This process can be performed for one, two, three, four, five, or more generations. At each generation including the F1 progeny, the first-generation selfed or sib-mated progeny, and all subsequent generations, the population can be screened for the desired characteristics, which screening can occur in a number of different ways. For instance, the population can be screened using phenotypic screens or quantitative bioassays as known in the art, which phenotypic screens or bioassays may be performed on individual plants of the population or on selfed or crossed progeny from the same individual plants. Alternatively, instead of using bioassays, marker-assisted selection can be performed to identify those plants that contain the autoflower trait and the Value Phenotype. Following screening, at each generation, one or several plants can be selected that contain the autoflower trait and the Value Phenotype as determined through either phenotypic or, in some embodiments, genotypic screening, and self-fertilized or sib-mated for a number of generations in order to allow for the cannabis plant to become increasingly similar to the Value Phenotype parent. This process can be performed for one, two, three, four, five, or more generations. In principle, the progeny resulting from the process of crossing the recurrent parent with the autoflower donor parent are heterozygous for one or more genes responsible for autoflowering. When appropriate, cycles of selfing or sib-mating and selection can be stopped, the last generation of selfed or sib-mated progeny consisting in a homozygous pure breeding (inbred) progeny with Autoflower Value Phenotype.

The result of backcrossing, selfing, sib-mating, and/or dihaploids, or any other technique is the production of lines that are genetically homogenous for the genes associated with autoflowering, and in some embodiments as well as for other genes associated with the Value Phenotype.

### EXAMPLES

### Example 1

### Phenotypic Correlation Between AF and Agronomic or Composition (Value Trait) Performance

Varieties extracted for commercial production were evaluated for different traits including, total cannabinoid concentration, total THC concentration, total terpene concentration (as mg/g of dry matter) and oil yield as % of fresh frozen biomass. Autoflower varieties showed significantly lower cannabinoid, THC and terpene concentrations, as well as oil yield than the daylength sensitive varieties.

TABLE 2
Sample descriptives for total concentration of cannabinoids, THC, terpenes and oil yield percent.

| | Cannabinoids Total | | THC Total | | Terpene Total | | Oil Yield | |
|---|---|---|---|---|---|---|---|---|
| | Concentration (mg/g) | | Concentration (mg/g) | | Concentration (mg/g) | | Percent (%) | |
| Class | AF | PP | AF | PP | AF | PP | AF | PP |
| # Materials | 214 | 341 | 214 | 341 | 216 | 154 | 33 | 155 |
| Mean | 134 | 207.5 | 121.7 | 183.1 | 3 | 5.4 | 4 | 5.9 |
| Std. Deviation | 31.8 | 35.5 | 30.5 | 31.8 | 1.4 | 2.5 | 0.5 | 0.9 |
| P value | | <0.001 | | <0.001 | | <0.001 | | <0.001 |

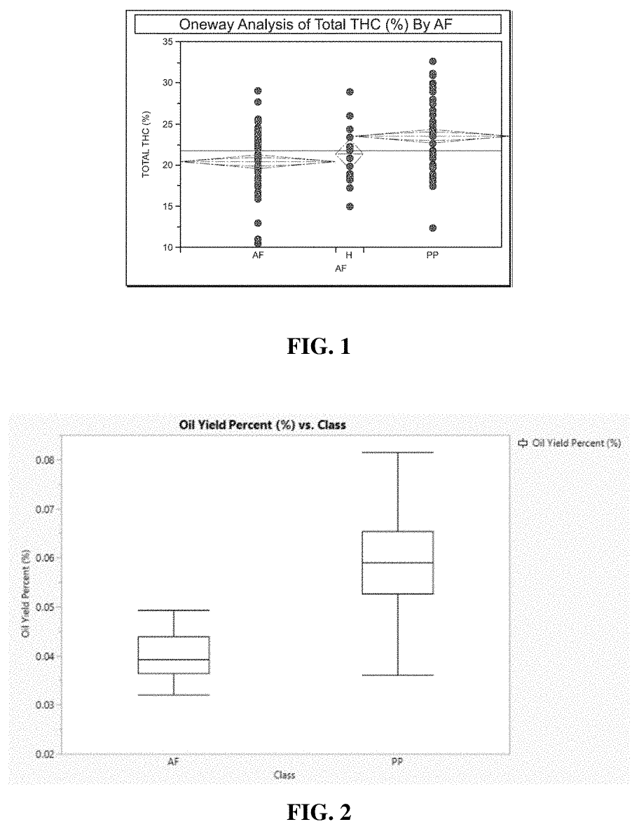

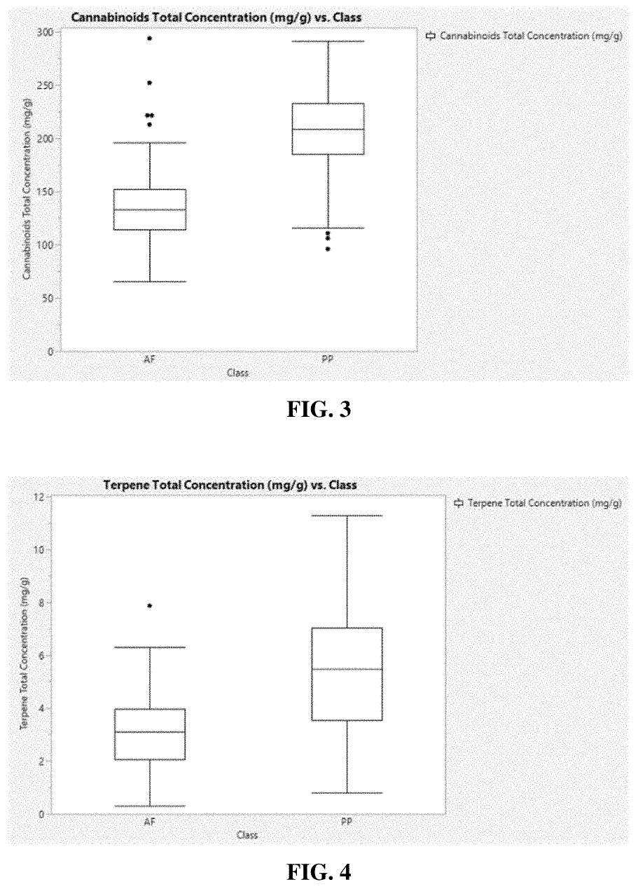

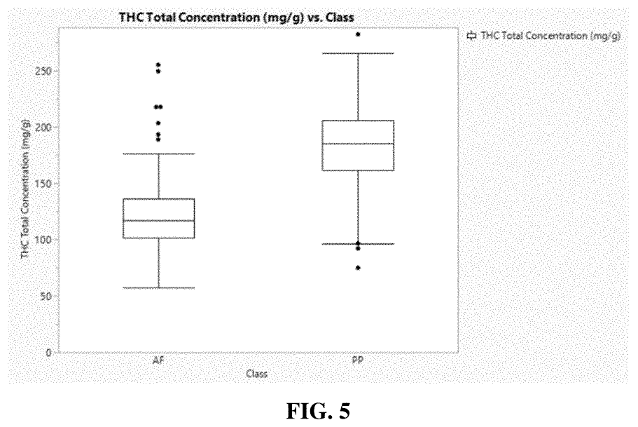

These results (summarized in Tables 3-5 and FIGS. 2-5) show the relationship between auto-flowering/daylength sensitivity and economically important traits in Cannabis sativa. The auto-flowering characteristic is always/generally associated with lower values of these economically important traits than daylength sensitivity. Because of the genetic structure of these two groups of materials—being selfed progenies of auto-flowering×daylength sensitive segregating crosses—this observation is strong evidence for the existence of negative genetic linkage between the AF allele at the autoflower locus and agronomically and economically desirable traits. Breaking such negative linkage will require specific processes, including the use of specific markers outside of, yet closely flanking, the AF locus.

### Example 2

### QTL Mapping of Autoflower and Agronomic and Composition Traits (Value Traits)

A population of 186 F2 Cannabis sativa plants was generated from a cross between a known photoperiod sensitive (PP) parent and a known photoperiod insensitive/autoflower (AF) parent to conduct a QTL mapping experiment for a number of traits of interest.

Each F2 plant was phenotyped in 2021 for daylength sensitivity (with two phenotypes: PP or AF), CBD content, THC content, and a number of other traits.

Each F2 plant was also genotyped at 600 SNP loci, including one marker very tightly linked to the AF/PP locus on chromosome 1 and fully diagnostic of the daylength sensitivity phenotype (AF marker). A QTL mapping analysis was conducted from the phenotypic and genotypic data, using single-factor analyses of variance (ANOVA), performed with JMP®, Version 16.1.0. SAS Institute Inc., Cary, N.C., 1989-2021.

A number of ANOVAs were found to be significant, including that where the dependent variable (phenotype) was THC content (%) and the independent variable (genotype) was the AF marker: (F(2,183)=16.064, p=<0.0001), the allele coming from the AF parent of the cross displaying a significantly lower THC content than the allele coming from the PP parent of that same cross. This evidence of the presence of a THC content QTL in the vicinity of the AF locus, in repulsion with the AF allele (unfavorable THC content allele in coupling with favorable daylength sensitivity allele), contributes to the understanding of the basis for the generally lower performance of AF germplasm when compared to PP germplasm, and sheds light on the fact that some of that difference in performance may be due to unfavorable linkages between AF and other traits, such as THC content as demonstrated here, on chromosome 1. See FIG. 1.

TABLE 3

| | |
|---|---|
| Rsquare | 0.149343 |
| Adj Rsquare | 0.140047 |
| Root Mean Square Error | 3.618427 |
| Mean of Response | 21.81034 |
| Observations (or Sum Wgts) | 186 |

TABLE 4

| | | Sum of | Mean | | |
|---|---|---|---|---|---|
| Source | DF | Squares | Square | F Ratio | Prob > F |
| AF | 2 | 420.6514 | 210.326 | 16.064 | <.0001 |
| Error | 183 | 2396.022 | 13.093 | | |
| C. Total | 185 | 2816.673 | | | |

TABLE 5

| | | | | Power | Upper |
|---|---|---|---|---|---|
| Level | Number | Mean | Std Error | 95% | 95% |
| AF | 90 | 20.3805 | 0.38142 | 19.628 | 21.133 |
| H | 16 | 21.3228 | 0.90461 | 19.538 | 23.108 |
| PP | 80 | 23.5164 | 0.40455 | 22.718 | 24.315 |

### Example 3

### AF Cultivar with High THCA

A breeding protocol is undertaken to develop an Autoflower Value Phenotype cultivar, specifically, a cultivar that combines the AF phenotype with a Value Trait (VT) characterized by high THCA accumulation. High THCA accumulation was determined as THCA levels in various groups having greater than 20%, 22%, 24%, 26%, 28%, or 30% weight percent dry flower. The protocol is initiated by selecting an AF parent and a VT parent whose phenotype for high THCA accumulation meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait. In order to obtain valid comparisons of THCA accumulation, a defined THCA quantification approach is selected from among various options, and is consistently used to compare VT and AF parents and all progeny. Likewise, consistency for comparison purposes is achieved by randomizing plants selected for sampling from a population of plants and by defining which portion of the flower of each plant to sample.

A breeding protocol is undertaken to develop an Autoflower Value Phenotype cultivar, specifically, a cultivar that combines the AF phenotype with a Value Trait (VT) characterized by high THCA accumulation. High THCA accumulation was determined as THCA levels in various groups having greater than 20%, 22%, 24%, 26%, 28%, or 30% weight percent dry flower. The protocol is initiated by selecting an AF parent and a VT parent whose phenotype for high THCA accumulation meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait. In order to obtain valid comparisons of THCA accumulation, a defined THCA quantification approach is selected from among various options, and is consistently used to compare VT and AF parents and all progeny. Likewise, consistency for comparison purposes is achieved by randomizing plants selected for sampling from a population of plants and by defining which portion of the flower of each plant to sample.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined within this Example, above.

In another sub-protocol of this Example, a feminized line of seeds new AFVT cultivar is obtained by conventional approaches to feminization of Cannabis cultivars.

### Example 4

### AF Cultivar with Desirable Terpenes and Aromatics

A breeding protocol is undertaken to develop an Autoflower Value Phenotype cultivar, specifically, a cultivar that combines the AF phenotype with a Value Trait (VT) characterized by having desirable terpenes and aromatics as described in the detailed description. The protocol is initiated by selecting an AF parent and a VT parent whose phenotype for having desirable terpenes and aromatics meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined within this Example, above.

In another sub-protocol of this Example, a feminized line of seeds new AFVT cultivar is obtained by conventional approaches to feminization of Cannabis cultivars.

TABLE 6
Examples of desirable/unique terpenes not
found in public AFs in mg/g fresh weight.

| | | | | Number of |
|---|---|---|---|---|
| Name | Eucalyptol | Geraniol | Sabinene | Samples |
| 20AF12412 | 0.082 | ND | ND | 1 |
| 20AF13800 | 0.066 | ND | ND | 1 |
| 20AF13601 | 0.068 | ND | ND | 1 |
| 20AF12493 | 0.360 | ND | 4.602 | 1 |
| 20AF15374 | ND | 0.399 | ND | 1 |
| 20AF13849 | 0.262 | ND | ND | 1 |
| 20AF15167 | ND | 0.181 | ND | 1 |
| 20AF15798 | 0.073 | 0.177 | 0.123 | 1 |
| 20AF15914 | ND | ND | 0.114 | 1 |
| 20AF15921 | ND | ND | 0.105 | 1 |
| 20AF15357 | ND | 0.340 | ND | 1 |
| Generic AF | ND | ND | ND | 66 |

TABLE 7
Total % terpene

| Name | Total Terpene (%) | Number of samples |
|---|---|---|
| 20AF17851 | 4.61 | 1 |
| 20AF17841 | 4.14 | 1 |
| 20AF17189 | 4.45 | 1 |
| 20AF17912 | 4.26 | 1 |
| 20AF13601 | 4.35 | 1 |
| 20AF13849 | 5.30 | 1 |
| 20AF16873 | 4.67 | 1 |
| Generic AF | 2.29 | 66 |

### Example 5

### AF Cultivar with High Biomass Yield

A breeding protocol is undertaken to develop an Autoflower Value Phenotype cultivar, specifically, a cultivar that combines the AF phenotype with a Value Trait (VT) characterized by high biomass yield as described in the detailed description. The protocol is initiated by selecting an AF parent and a VT parent whose phenotype for high biomass yield meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined within this Example, above, and specifically when biomass yield is at or above 2 pounds per plant (wet weight) or 20,000 pounds (wet weight) per acre.

In another sub-protocol of this Example, a feminized line of seeds new AFVT cultivar is obtained by conventional approaches to feminization of Cannabis cultivars.

### Example 6

### AF Cultivar with Desirable Biomass Composition

A breeding protocol is undertaken to develop an Autoflower Value Phenotype cultivar, specifically, a cultivar that combines the AF phenotype with a Value Trait (VT) characterized by having a desirable biomass composition as described in the detailed description. The protocol is initiated by selecting an AF parent and a VT parent whose phenotype for a desirable biomass composition meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined within this Example, above.

In another sub-protocol of this Example, a feminized line of seeds new AFVT cultivar is obtained by conventional approaches to feminization of Cannabis cultivars.

### Example 7

### AF Cultivar with High Crude Oil Yield

A breeding protocol is undertaken to develop an Autoflower Value Phenotype cultivar, specifically, a cultivar that combines the AF phenotype with a Value Trait (VT) characterized by having high crude oil yield as defined in the detailed description. The protocol is initiated by selecting an AF parent and a VT parent whose phenotype for high crude oil yield meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined within this Example, above.

In another sub-protocol of this Example, a feminized line of seeds new AFVT cultivar is obtained by conventional approaches to feminization of Cannabis cultivars.

TABLE 8
Exemplary crude oil yield.

| Genotype | Crude Oil Yield (%) | Number of samples |
|---|---|---|
| 19AFB086 | 4.55 | 1 |
| 19AFB085 | 4.53 | 1 |
| 19AFB091 | 4.73 | 1 |
| Generic AF | 3.15 | 86 |

### Example 8

### AF Cultivar with Desirable Crude Oil Composition

A breeding protocol is undertaken to develop an Autoflower Value Phenotype cultivar, specifically, a cultivar that combines the AF phenotype with a Value Trait (VT) characterized by having a desirable crude oil composition as described in the detailed description herein. The protocol is initiated by selecting an AF parent and a VT parent whose phenotype for a desirable crude oil composition meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined within this Example, above.

In another sub-protocol of this Example, a feminized line of seeds new AFVT cultivar is obtained by conventional approaches to feminization of Cannabis cultivars.

### Example 9

### AF Cultivar with Modulators of Flowering Time

A breeding protocol is undertaken to develop an Autoflower Value Phenotype cultivar, specifically, a cultivar that combines the AF phenotype with a Value Trait (VT) characterized by having modulators of flowering time as described in the detailed description. The protocol is initiated by selecting an AF parent and a VT parent whose phenotype for having modulators of flowering time meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined within this Example, above.

In another sub-protocol of this Example, a feminized line of seeds new AFVT cultivar is obtained by conventional approaches to feminization of Cannabis cultivars.

### Example 10

### AF Cultivar with a Desirable Flower to Leaf Ratio

A breeding protocol is undertaken to develop an Autoflower Value Phenotype cultivar, specifically, a cultivar that combines the AF phenotype with a Value Trait (VT) characterized by having a desirable flower to leaf ratio as described in the detailed description. The protocol is initiated by selecting an AF parent and a VT parent whose phenotype for a desirable flower to leaf ratio meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined within this Example, above.

In another sub-protocol of this Example, a feminized line of seeds new AFVT cultivar is obtained by conventional approaches to feminization of Cannabis cultivars.

### Example 11

### AF Cultivar with a Desirable Flower Structure

A breeding protocol is undertaken to develop an Autoflower Value Phenotype cultivar, specifically, a cultivar that combines the AF phenotype with a Value Trait (VT) characterized by having a desirable flower structure as described in the detailed description. The protocol is initiated by selecting an AF parent and a VT parent whose phenotype for a desirable flower structure meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined within this Example, above.

In another sub-protocol of this Example, a feminized line of seeds new AFVT cultivar is obtained by conventional approaches to feminization of Cannabis cultivars.

### Example 12

### AF Cultivar with High THCa Accumulation and a Desirable Flower Structure

A breeding protocol is undertaken to develop an Autoflower Value Phenotype cultivar, specifically, a cultivar that combines the AF phenotype with a Value Trait (VT) characterized by high THCa accumulation and a desirable flower structure. The protocol is initiated by selecting an AF parent and a VT parent whose phenotype for high THCa accumulation and a desirable flower structure meets the criteria specified in Examples 3 and 11. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined in Examples 3 and 11.

In another sub-protocol of this Example, a feminized line of seeds new AFVT cultivar is obtained by conventional approaches to feminization of Cannabis cultivars.

### Example 13

### AF Cultivar with High THCa Accumulation and Desirable Terpenes

A breeding protocol is undertaken to develop an Autoflower Value Phenotype cultivar, specifically, a cultivar that combines the AF phenotype with a Value Trait (VT) characterized by high THCa accumulation and desirable terpenes. The protocol is initiated by selecting an AF parent and a VT parent whose phenotype for high THCa accumulation and desirable terpenes meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined in Examples 3 and 4.

In another sub-protocol of this Example, a feminized line of seeds new AFVT cultivar is obtained by conventional approaches to feminization of Cannabis cultivars.

TABLE 9
Exemplary terpene and THC concentrations

| | Total Terpene (%) | Total THC (%) | Number of |
|---|---|---|---|
| Genotype | Mean | Mean | samples |
| 20AF17851 | 4.61 | 11.79 | 1 |
| 20AF17189 | 4.45 | 12.54 | 1 |
| 20AF17912 | 4.26 | 10.88 | 1 |
| 20AF13601 | 4.35 | 11.95 | 1 |
| 20AF13849 | 5.30 | 12.34 | 1 |
| 20AF16873 | 4.67 | 14.79 | 1 |
| Generic AF | 2.29 | 9.09 | 66 |

### Example 14

### AF Cultivar with High Crude Oil Yield and Biomass Yield

A breeding protocol is undertaken to develop an Autoflower Value Phenotype cultivar, specifically, a cultivar that combines the AF phenotype with a Value Trait (VT) characterized by high crude oil yield and biomass yield. The protocol is initiated by selecting an AF parent and a VT parent whose phenotype for high crude oil yield and biomass yield meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined in Examples 5 and 7.

In another sub-protocol of this Example, a feminized line of seeds new AFVT cultivar is obtained by conventional approaches to feminization of Cannabis cultivars.

### Example 15

### AF Cultivar with High Crude Oil Yield and Modulators of Flowering Time

A breeding protocol is undertaken to develop an Autoflower Value Phenotype cultivar, specifically, a cultivar that combines the AF phenotype with a Value Trait (VT) characterized by high crude oil yield and modulators of flowering time. The protocol is initiated by selecting an AF parent and a VT parent whose phenotype for high crude oil yield and modulators of flowering time meets the criteria specified in this Example. These parent plants are crossed and F1 progeny are recovered. In “sub-protocol A”, F1 progeny are screened for presence of an AF marker for further breeding. In “sub-protocol B” random members of the F1 population are crossed or selfed. In either case, F2 progeny are obtained and evaluated phenotypically for presence of the AF trait and the Value Trait.

Further rounds of crosses and selections are performed employing approaches well established in the art of plant breeding. In sub-protocol A, selections include genotyping progeny in one or more generations for presence or absence of relevant markers including but not limited to AF markers. In sub-protocol B, all selections are phenotypic.

Successive backcrosses and other conventional approaches to interbreeding are performed with selection criteria including the intra-generational uniformity and inter-generations stability of the VT in combination with consistent, essentially uniform manifestation of the AF trait. The breeding protocol is defined as complete when a novel, stable cultivar is produced whose seed is >99% AF and is within 70% of all VT criteria as defined in Examples 7 and 9.

In another sub-protocol of this Example, a feminized line of seeds new AFVT cultivar is obtained by conventional approaches to feminization of Cannabis cultivars.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A Cannabis plant or plant part with an Autoflower Value Phenotype, wherein the Autoflower Value Phenotype is seed-propagated, stable and uniformly expressed, wherein the Autoflower Value Phenotype comprises at least one value trait selected from:
a. high THCA accumulation;
b. specific cannabinoid ratio(s);
c. a desirable composition of terpenes and/or other aromatic molecules;
d. biomass yield;
e. biomass composition;
f. crude flower oil yield;
g. crude flower oil composition;
h. specific variants affecting cannabinoid or aromatic molecule biosynthetic pathways;
i. a finished plant height that enables tractor farming inside high tunnels;
j. modulators of the flowering time phenotype that increase or decrease maturation time;
k. high flower to leaf ratios that enable pathogen resilience through improved airflow;
l. high flower to leaf ratios that maximize light penetration and flower development in the vertical canopy space;
m. a finished plant height and flower to leaf ratio that maximizes light penetration all the way to the ground but minimizes total plant height; and/or
n. advantageous flower structures for oil or flower production
i. flower diameter,
ii. flower length,
iii. flower density,
iv. long or short internodal spacing distance,
v. flower-to-leaf determination ratio of flower tissue (leafiness of flower), and/or
vi. uniform size, shape, and density of flowers throughout a plant.

2. The plant of claim 1, wherein the plant has at least two value traits.

3. The plant of claim 1, wherein the plant has at least three value traits.

4. The plant of claim 1, wherein a quantitative level of the value trait is at least 70% of the value trait present in a photoperiod parent.

5. The plant of claim 1, wherein a quantitative level of the value trait has not previously been available in seed-propagated autoflower Cannabis displaying substantial uniformity and stability as a seed line.

6. The plant of claim 1, wherein the value trait is high THCA accumulation, and wherein the plant has THCA levels greater than 20%.

7. The plant of claim 1, wherein the plant comprises improved crude oil yield, wherein the crude oil yield is at least 4.5%.

8. The plant of claim 1, wherein the plant comprises an advantageous plant structure, wherein the advantageous plant structure permits a light measurement at a given position, and/or a total light measurement of all positions, at least 10% greater than a reference autoflower parent plant.

9. The plant of claim 1, wherein the plant comprises high THCA accumulation and advantageous flower structure.

10. The plant of claim 1, wherein the plant comprises high THCA accumulation and desirable terpene composition.

11. The plant of claim 1, wherein the plant comprises high crude oil yield and high biomass yield.

12. A method of plant breeding to develop the plant of claim 1, comprising
a. providing a first parent plant having a phenotype defined as a Value Phenotype, wherein the Value Phenotype comprises at least one trait of interest;
b. providing a second parent plant, having an autoflower phenotype;
c. crossing the first and second parent plants;
d. recovering progeny from the crossing step;
e. screening the progeny phenotypically for presence of at least one autoflower allele and the Value Phenotype;
f. selecting autoflower carrier progeny with the Value Phenotype, wherein cells of said autoflower carrier progeny comprise at least one autoflower allele;
g. conducting further breeding steps using autoflower carrier progeny selfed, sib-mated, or crossed with plants having the Value Phenotype;
h. repeating steps e, f, and g until at least one plant having an Autoflower Value Phenotype is obtained.

13. A method of plant breeding to develop the plant of claim 1, comprising
a. providing a first parent plant, having a phenotype defined as a Value Phenotype, wherein the Value Phenotype comprises at least one trait of interest;
b. providing a second parent plant, having an autoflower phenotype;
c. crossing the first and second parent plants;
d. recovering progeny from the crossing step;
e. screening the progeny for presence of at least one autoflower allele using a marker having at least 51% correlation with presence of the autoflower allele; or without a marker by screening the progeny's selfed offspring for the ability to produce the homozygous autoflowering phenotypes;
f. selecting autoflower carrier progeny, wherein cells of said autoflower carrier progeny comprise at least one autoflower allele;
g. conducting further breeding steps using autoflower carrier progeny crossed with plants having the Value Phenotype;
h. repeating steps e, f, and g until at least one plant having an Autoflower Value Phenotype is obtained.

14. A method of plant breeding to develop the plant of claim 1, comprising
a. providing a first parent plant, having a phenotype defined as a Value Phenotype, wherein the Value Phenotype comprises at least one trait of interest;
b. providing a second parent plant, having an autoflower phenotype;
c. crossing the first and second parent plants;
d. recovering progeny from the crossing step;
e. screening the progeny for presence of at least one autoflower allele using a marker having at least 51% correlation with presence of the autoflower allele; or without a marker by screening the progeny's selfed offspring for the ability to produce the homozygous autoflowering phenotypes;
f. selecting autoflower carrier progeny, wherein cells of said autoflower carrier progeny comprise at least one autoflower allele;
g. conducting further breeding steps using autoflower carrier progeny crossed with plants having the Value Phenotype;
h. repeating steps e, f, and g until at least one plant having an Autoflower Value Phenotype is obtained.
